(19) United States
(12) Patent Application Publication
(10) Pub. No.: US 2026/0001909 A1
(43) Pub. Date: Jan. 1, 2026

(54) Method for Preparing a Peptide With CCK Receptor Modulatory Activity

(71) Applicant: CRMH, HONG KONG INSTITUTE OF SCIENCE & INNOVATION, CHINESE ACADEMY OF SCIENCES, Hong Kong (CN)

(72) Inventors: Mickey Daniel Tortorella, Hong Kong (CN); Huatang Zhang, Hong Kong (CN)

(21) Appl. No.: 19/253,716

(22) Filed: Jun. 28, 2025

(30) Foreign Application Priority Data
Jun. 28, 2024 (CN) 202410855537.X

(51) Int. Cl. C07K 5/117; A61K 38/00

(52) U.S. Cl. CPC C07K 5/1024; A61K 38/00

(57) ABSTRACT

The present invention discloses a method for preparing a peptide compound with CCK receptor agonistic and/or antagonistic activity. The method uses a sequence of solution-phase reactions to generate high-purity and scalable product without chromatographic purification, suitable for industrial production and pharmaceutical development.

### CROSS-REFERENCE TO RELATED APPLICATIONS

This application claims priority to Chinese Patent Application No. CN202410855537.X, filed on Jun. 28, 2024, entitled “A Method for Preparing a Polypeptide with CCK Receptor Agonistic/Antagonistic Activity”, the entire contents of which are incorporated herein by reference.

### FIELD

The present invention relates to the field of chemical and pharmaceutical technology, and in particular to a method for preparing peptides with cholecystokinin (CCK) receptor agonistic and/or antagonistic activity.

### BACKGROUND

Cholecystokinin (CCK) is a peptide hormone consisting of 33 amino acids, synthesized and secreted by cells in the duodenum and jejunum. It is widely distributed in the brain, especially in regions such as the cortex, striatum, hippocampus, pretectal area, septum, and hypothalamus. It also functions in the periphery as a neurotransmitter or neuromodulator via intestinal secretion.

CCK plays significant physiological roles, including promoting gallbladder contraction, stimulating pancreatic enzyme secretion, enhancing insulin release, increasing bile secretion, delaying gastric emptying, stimulating mucous secretion, promoting intestinal peristalsis, and inhibiting the absorption of potassium, sodium, chloride, and fluids in the jejunum and ileum.

Two subtypes of CCK receptors have been identified in both the central and peripheral nervous systems. Agonists and antagonists of CCK receptors have potential as therapeutic agents for conditions such as obesity, gallbladder cancer, pancreatic cancer, epilepsy, amblyopia, tinnitus, depression, and acid-related gastrointestinal disorders.

The prior art, such as WO2023065716A1, discloses peptides with high CCK receptor agonist/antagonist activity and improved half-life and efficacy compared to CCK-4. These compounds showed improvements in spatial memory deficits in aged and Alzheimer's disease mouse models.

However, in the prior art, only small amounts of the peptides were synthesized using solid-phase synthesis for activity screening purposes. This method is limited by low yield and laboratory scale, and is not suitable for preclinical CMC-stage production of peptide candidates.

In the preclinical phase of drug development, after identifying compounds with suitable activity and pharmacokinetic profiles through structure-activity relationship (SAR) studies, there is a need to establish scalable and industrially applicable synthetic methods. These methods must support the production of pharmaceutical-grade compounds, impurity research, quality studies, and stability evaluations.

### SUMMARY

The primary objective of the present invention is to overcome the limitations of prior art by providing a solution-phase method for preparing peptides with cholecystokinin (CCK) receptor agonistic and/or antagonistic activity. This method offers improved yield, avoids the need for column chromatography or HPLC purification, and is suitable for scale-up in pilot and industrial production.

In particular, the peptides prepared by this method exhibit dual pharmacological activity: acting as CCK receptor agonists at low concentrations and antagonists at high concentrations. The process provides high-purity target compounds that meet the requirements of pharmaceutical research and industrial manufacturing.

According to one aspect of the invention, a method for preparing a compound represented by compound 12 is provided, comprising the following steps:
Step I: Condensation of compound 03 with compound 04 to yield compound 05;
Step II: Deprotection of the amino protecting group of compound 05 to obtain compound 06;
Step III: Condensation of compound 06 with compound 07 to yield compound 08;
Step IV: Deprotection of the amino protecting group of compound 08 to obtain compound 09;
Step V: Condensation of compound 09 with compound 10 to obtain compound 11;
Step VI: Removal of the carboxyl protecting group of compound 11 to yield compound 12.

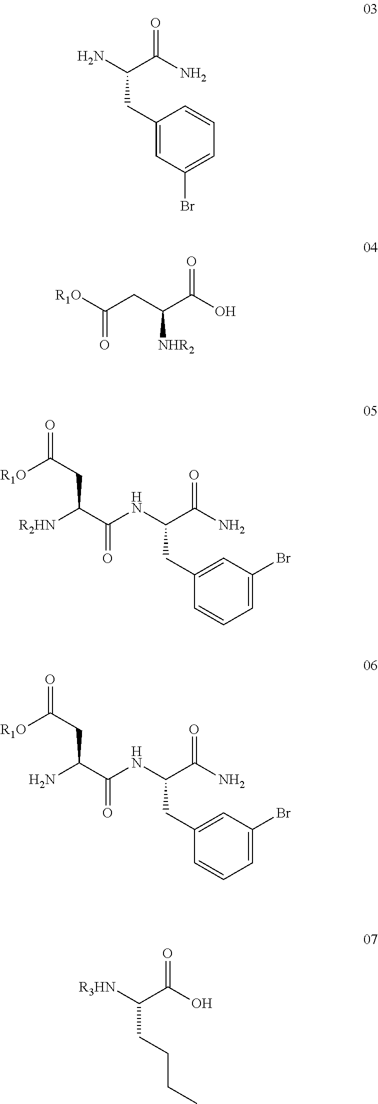

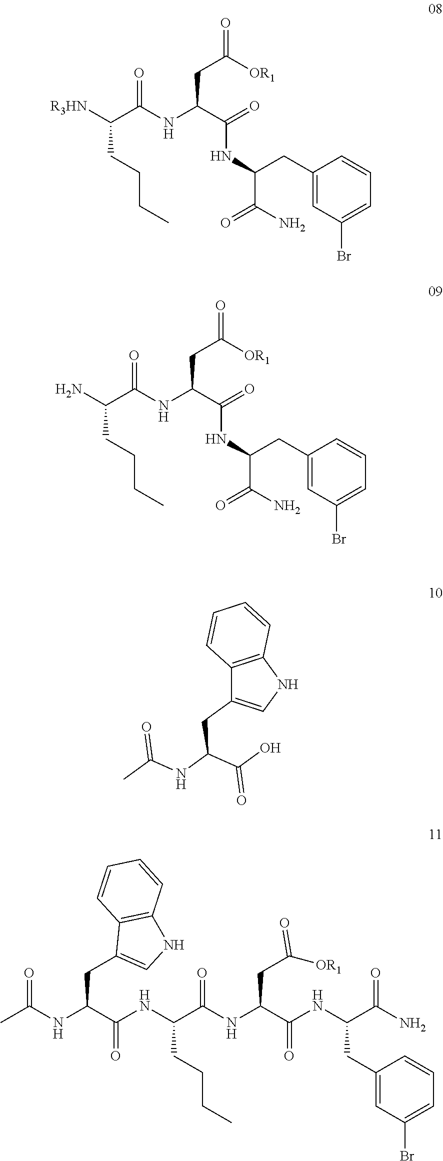

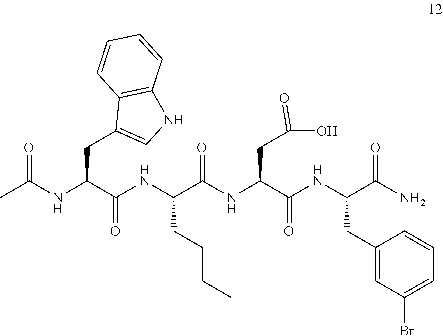 [SMILES: CCCC[C@H](NC(=O)[C@H](Cc1c[nH]c2ccccc12)NC(C)=O)C(=O)N[C@@H](CC(=O)O)C(=O)N[C@@H](Cc1cccc(Br)c1)C(N)=O]

The invention also encompasses pharmaceutical salts of compound 12, and further provides preferred reaction conditions, protecting group strategies, reagents, and solvents suitable for each reaction step, enabling scalable and reproducible manufacturing of the target peptides.

This synthetic approach achieves: (a) High chemical yield and optical purity; (b) Elimination of column purification; (c) Compatibility with preclinical CMC (Chemistry, Manufacturing, and Controls) needs.

### BRIEF DESCRIPTION OF THE DRAWINGS

The drawings appended to this application illustrate various analytical and structural data of the compounds synthesized in the examples:

FIG. 1: ¹H NMR spectrum of compound 02.

FIG. 2: Mass spectrum (MS) of compound 02.

FIG. 3: ¹H NMR spectrum of compound 03.

FIG. 4: MS of compound 03.

FIG. 5: ¹H NMR spectrum of compound 05A.

FIG. 6: MS of compound 05A.

FIG. 7: ¹H NMR spectrum of compound 06A.

FIG. 8: MS of compound 06A.

FIG. 9: ¹H NMR spectrum of compound 08A.

FIG. 10: MS of compound 08A.

FIG. 11: ¹H NMR spectrum of compound 09A.

FIG. 12: MS of compound 09A.

FIG. 13: ¹H NMR spectrum of compound 11A.

FIG. 14: MS of compound 11A.

FIG. 15: ¹H NMR spectrum of compound 12.

FIG. 16: MS of compound 12.

These figures collectively demonstrate the structure, purity, and molecular integrity of the intermediate and final compounds obtained through the described synthetic route.

### DETAILED DESCRIPTION

The present invention provides a solution-phase synthetic method for preparing peptides with cholecystokinin (CCK) receptor agonistic and/or antagonistic activity. This method is characterized by high yield, mild reaction conditions, and operational simplicity without chromatographic purification. The synthetic strategy employs stepwise peptide coupling reactions combined with protecting group manipulations to yield the target compound, which can be used in pharmaceutical research and development.

Unless otherwise defined, all technical and scientific terms used in this document have the same meanings as those commonly understood by those skilled in the art belonging to the technical field of this application. The terms used in the description of this application in the specification of this application are merely for the purpose of describing specific embodiments and are not intended to limit this application. The term “and/or” used herein includes any and all combinations of one or more of the listed items.

The following further explains this application in conjunction with the drawings and specific embodiments, so that those skilled in the art can better understand this application and implement it, but the examples given are not intended to limit this application.

The experimental methods used in the following embodiments, unless otherwise specified, are all conventional methods. The materials and reagents used, unless otherwise specified, can all be obtained from commercial sources.

### EXAMPLES

The following abbreviations are used throughout this specification:
DCC: N,N′-Dicyclohexylcarbodiimide
HOBt: 1-Hydroxybenzotriazole
EtOAc: Ethyl acetate
DMF: N,N-Dimethylformamide
DCM: Dichloromethane
TFA: Trifluoroacetic acid
NaHCO₃: Sodium bicarbonate
EDCI: 1-Ethyl-3-(3-dimethylaminopropyl) carbodiimide
DIEA: N,N-Diisopropylethylamine
Me₃SiI: Trimethylsilyl iodide
Na₂S₂O₃: Sodium thiosulfate

The synthetic method comprises the following steps:

### Deprotection Process I:

The N-terminal protecting group of compounds 03, 06B, 06D, 09B and 09D in example 2 and example 4 is removed using HCl in EA.

### Deprotection Process II:

The N-terminal protecting group of compounds 06A from 05C and 09A from 08C in example 3 is removed using Me₃SiI in DCM.

The following examples further illustrate the invention without limiting its scope.

The synthetic route comprises stepwise coupling and deprotection of intermediates to yield the final peptide compound without chromatographic purification, which exhibits CCK receptor modulatory activity.

### Example 1: Preparation of Compound 12 with Synthesis Route A

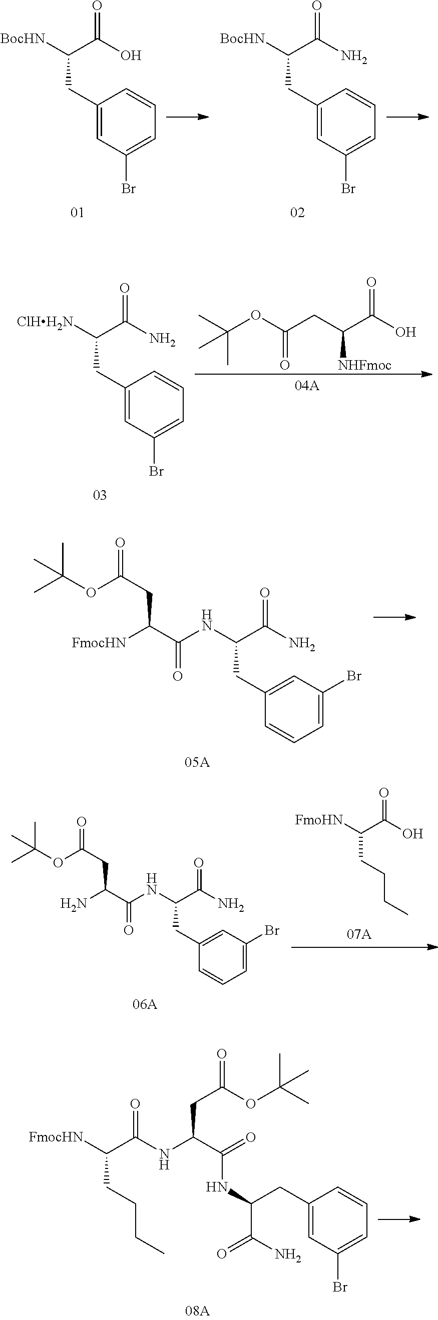

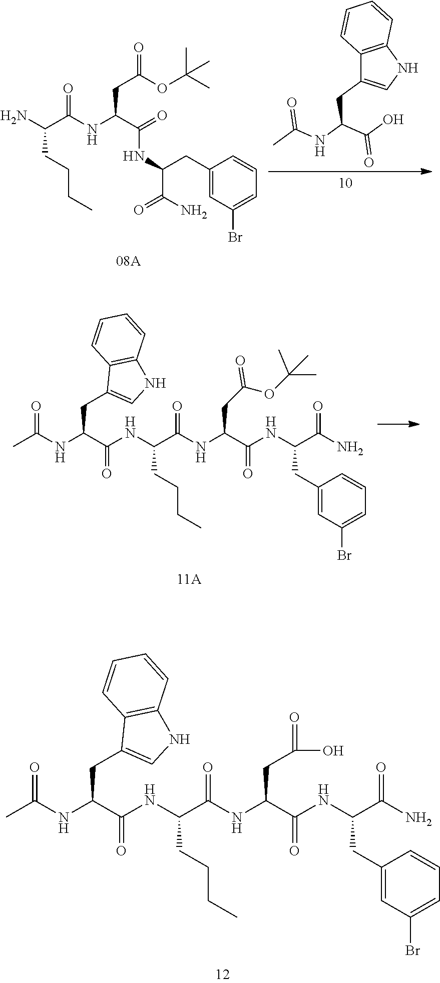

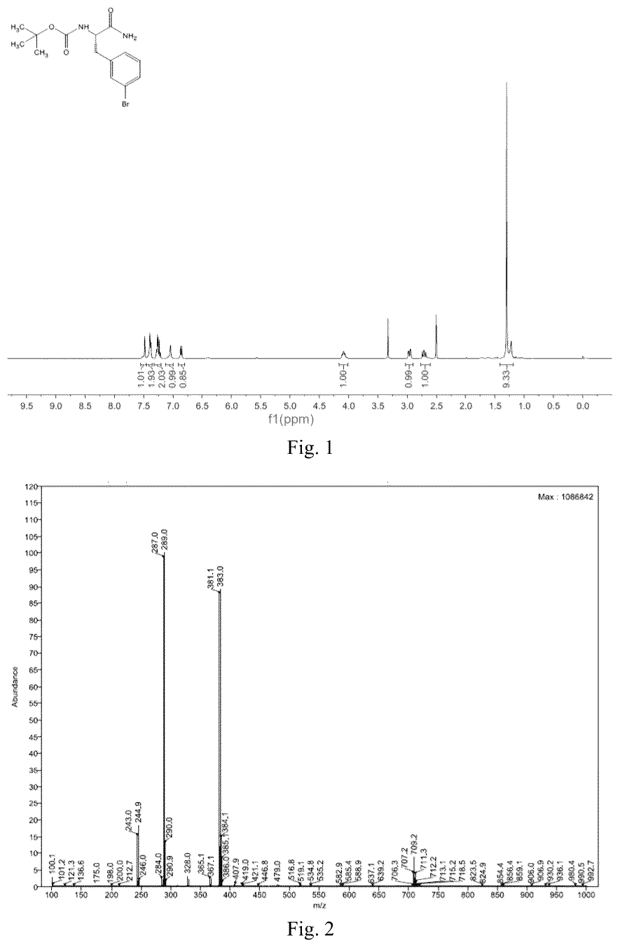

Compound 01 (300 g, 1.0 eq), HOBt (177 g, 1.5 eq), EDCI (167 g, 1.0 eq) and DMF (2100 mL, 7 v) were added to a 10 L jacketed flask, and nitrogen was purged twice. The mixture was cooled to −5° C., and ammonia water (107 g, 1.8 eq) was slowly added at this temperature. After the system was warmed by 2° C., the addition was completed. Then, the mixture was stirred at −5° C. for 17 hours under HPLC monitoring. The reaction solution was slowly poured into water (10 mL, 35 v), and after stirring for 3 hours, it was filtered. The filter cake was dissolved in ethyl acetate (6 L, 20 v), dried with anhydrous sodium sulfate; the solution was evaporated under reduced pressure to obtain compound 02, a white powder, 270 g; purity 99%. The ¹H NMR spectrum of compound 02 is shown in FIG. 1, the MS spectrum is shown in FIG. 2, ¹H NMR (DMSO-d⁶): 7.48 (1H, s), 7.38 (2H, d), 7.25 (2H, m), 7.04 (1H, s), 6.86 (1H, d), 4.08 (1H, td), 2.96 (1H, dd), 2.70 (1H, dd), 1.30 (9H, s); ESI-MS: [M+K]⁺: 381.1, 383.0.

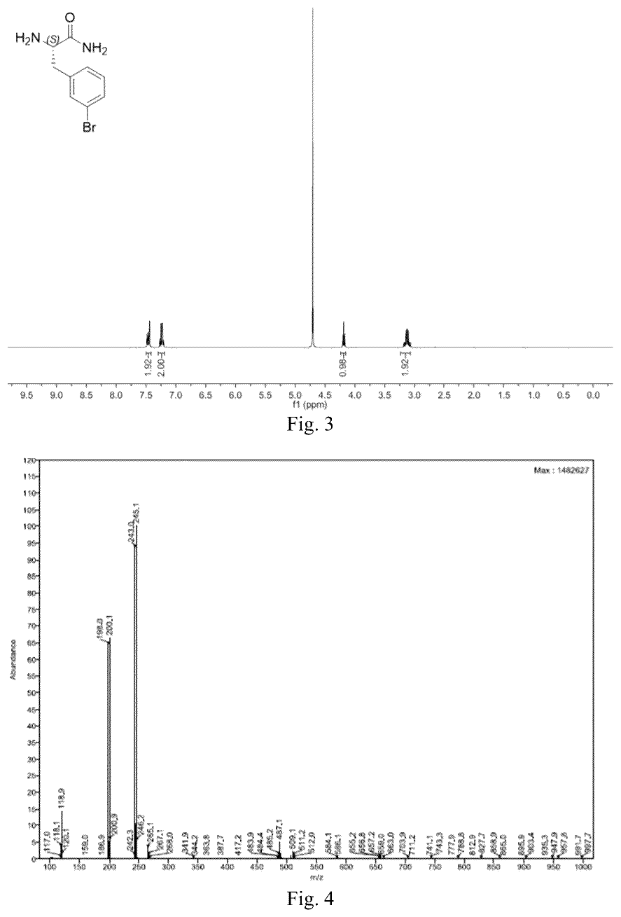

In a 5 L reaction flask, compound 02 (300 g, 1 eq) and ethyl acetate (3 L, 10V) were added, followed by the addition of 3M ethyl acetate hydrochloric acid solution (1.5 L, 5V). The mixture was stirred at 20° C. for 2 hours until the reaction was complete. The reaction solution was filtered, and the filter cake was washed with ethyl acetate (2V), then dried under vacuum. The dried product was obtained, which was a white powder, weighing 194 g; the purity was 97.27%. The ¹H NMR spectrum of compound 03 is shown in FIG. 3, the MS spectrum is shown in FIG. 4, ¹H NMR (D₂O): 7.56 (1H, dt), 7.53 (1H, s), 7.32 (m, 2H), 4.27 (1H, t), 3.21 (2H, qd); ESI-MS: [M+H]⁺: 243.0, 245.1.

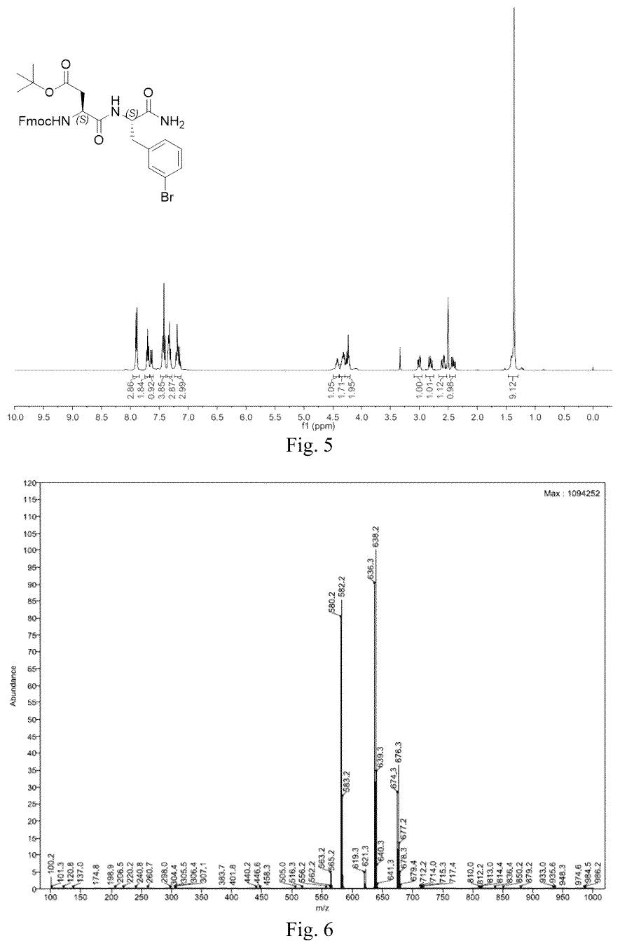

Compound 03 (145.3 g, 1.1 equivalents) and N,N-dimethylformamide (7 v) were added to a three-necked flask, cooled to 0° C. under an ice bath. N-methylmorpholine (2.0 equivalents) and(S)-4-(tert-butoxycarbonyl)-2-((tert-butoxycarbonyl)amino)-4-oxobutanoic acid (04A) (213.4 g, 1.0 equivalent) were added to the reaction flask and stirred at 0° C. for 0.5 hours; then HOBt (1.5 equivalents) and EDCI (1.1 equivalents) were added. The reaction mixture was stirred at 20° C. for 1 hour under HPLC control. The reaction solution was slowly poured into water (7 v), stirred for 1 hour, and filtered. The filter cake was hydrated with water (3 v) for 1 hour; the solid was dried to obtain compound 05A, a white powder, 345.9 g, with a purity of 97.3%. The ¹H NMR spectrum of compound 05A is shown in FIG. 5, the MS spectrum is shown in FIG. 6, ¹H NMR (DMSO-d⁶): 7.89 (3H, d), 7.7 (2H, t), 7.63 (1H, d), 7.42 (4H, m), 7.32 (3H, m), 7.17 (3H, m), 4.41 (1H, m), 4.32 (2H, m), 4.23 (2H, m), 3.00 (1H, dd), 2.80 (1H, dd), 2.59 (1H, dd), 2.41 (1H, dd), 1.36 (9H, s); ESI-MS: [M+H]+: 636.3, 638.2.

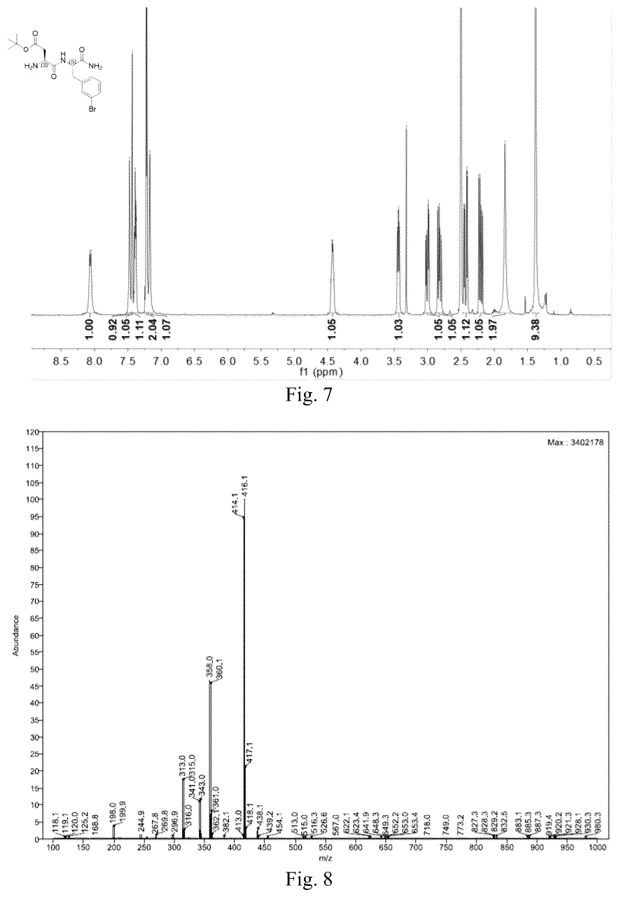

Add compound 05A (1200 g, 1.0 eq) and dichloromethane (7V) to the three-necked flask. Cool the mixture to 0° C. under an ice bath and then dropwise add piperidine (0.5 V). Stir at 0° C. for 1 hour. Monitor the reaction by TLC plate/HPLC until it is complete. Add n-heptane (7V) to the reaction mixture and homogenize for 1 hour; filter, wash the filter cake with n-heptane (2V), and filter. Dry the filter cake under vacuum to obtain compound 06A, a white solid, 567.5 g; purity 98.25%. The ¹H NMR spectrum of compound 06A is shown in FIG. 7, the MS spectrum is shown in FIG. 8, ¹H NMR (DMSO-d⁶): 8.06 (1H, d), 7.48 (1H, s), 7.43 (1H, s), 7.39 (1H, m), 7.22 (2H, d), 7.17 (1H, s), 4.43 (1H, dd), 3.44 (1H, dd), 3.00 (1H, dd), 2.82 (1H, dd), 2.43 (1H, dd), 2.20 (1H, dd), 1.84 (2H, s), 1.38 (9H, s); ESI-MS: [M+H]+: 414.1, 416.1.

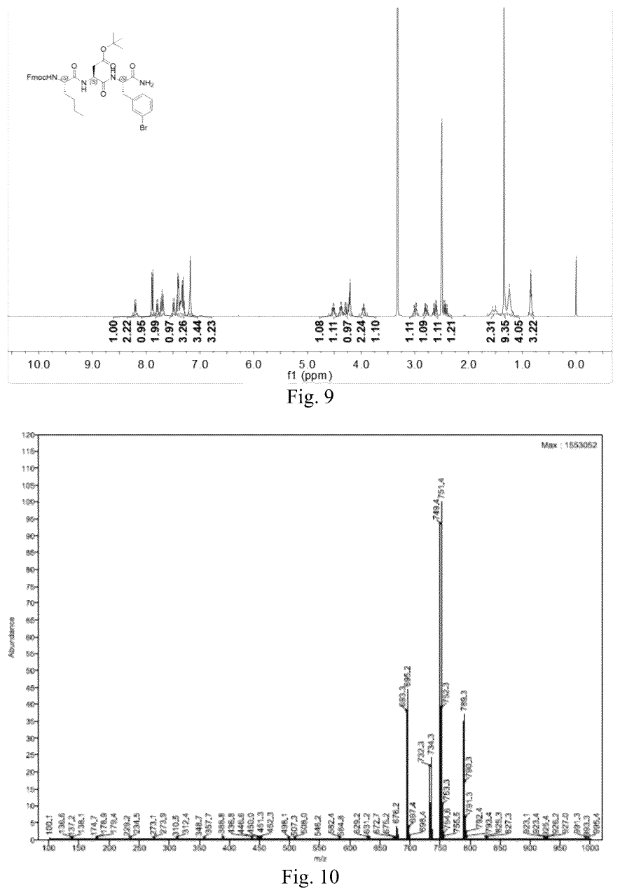

Add compound 06A (567.5 g, 1.0 eq), DMF (7 v), into a 250 mL jacketed flask and purify with nitrogen twice; add N-methylmorpholine (3.0 eq) and(S)-2-((((9H-fluorene-9-yl) methoxy) carbonyl) amino) heptanoic acid (07A) (484.1 g, 1.0 eq) into the reaction flask and stir at 0° C. for 30 min. Then add HOBt (1.5 eq), EDCI (1.1 eq); stir at 20° C. for 16 hours, control by HPLC; slowly pour the reaction liquid into water (10 v), stir for 2 hours; filter, wash the filter cake with water (2v); dry the solid, obtaining compound 08A, white solid, 785.6 g; purity 97.3%. The ¹H NMR spectrum of compound 08A is shown in FIG. 9, the MS spectrum is shown in FIG. 10, ¹H NMR (DMSO-d⁶): 8.21 (1H, d), 7.89 (2H, d), 7.79 (1H, d), 7.70 (2H, d), 7.49 (1H, d), 7.41 (3H, m), 7.32 (3H, m), 7.18 (3H, m), 4.52 (1H, q), 4.37 (1H, m), 4.29 (1H, m), 4.22 (2H, m), 3.95 (1H, q), 2.99 (1H, dd), 2.79 (1H, dd), 2.62 (1H, dd), 2.43 (1H, dd), 1.52 (2H, m), 1.34 (9H, s), 1.24 (4H, m), 0.84 (3H, t); ESI-MS: [M+H]⁺: 749.4, 751.4.

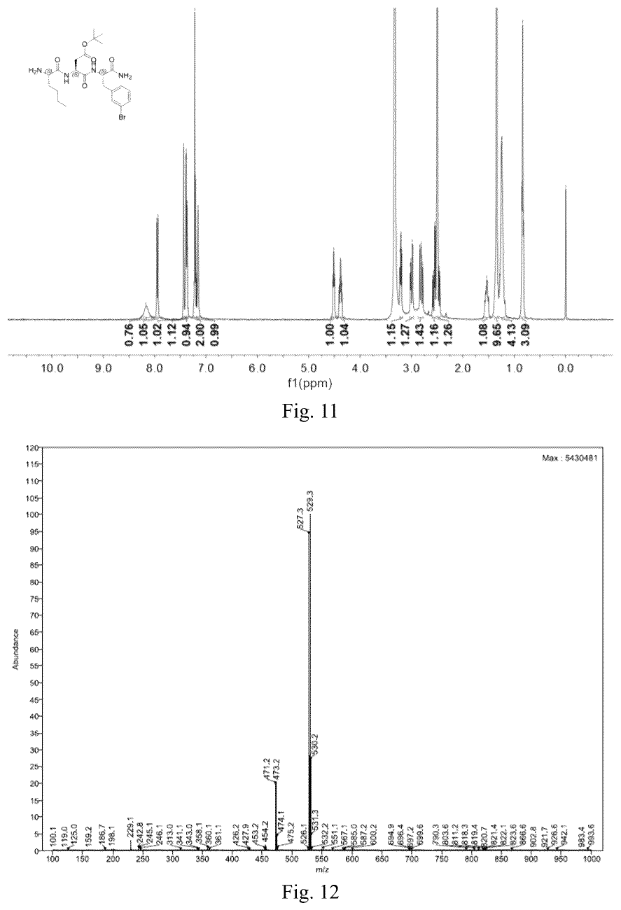

Add compound 08A (200 g, 1.0 eq) to the three-necked flask; add DCM (7V) and cool it to 0° C.; dropwise add piperidine (0.5 V) and stir at 0° C. for 1 hour; monitor the reaction with TLC/HPLC until completion; add n-hexane (7V) to the reaction mixture and homogenize for 1 hour; filter, wash the filter cake with n-hexane (10V), and filter again; dry the filter cake under vacuum to obtain compound 09A, a white solid, 133.96 g; purity 98.3%. The ¹H NMR spectrum of compound 09A is shown in FIG. 11, the MS spectrum is shown in FIG. 12, ¹H NMR (DMSO-d⁶): 8.17 (1H, s), 7.95 (1H, s), 7.44 (1H, s), 7.39 (1H, d), 7.37 (1H, m), 7.22 (2H, m), 7.15 (1H, s), 4.51 (1H, t), 4.38 (1H, m), 3.21 (1H, dd), 3.00 (1H, dd), 2.81 (1H, dd), 2.56 (1H, dd), 2.46 (1H, dd), 1.54 (1H, m), 1.34 (10H, s), 1.24 (4H, m), 0.84 (3H, t); ESI-MS: [M+H]⁺: 527.3, 529.3.

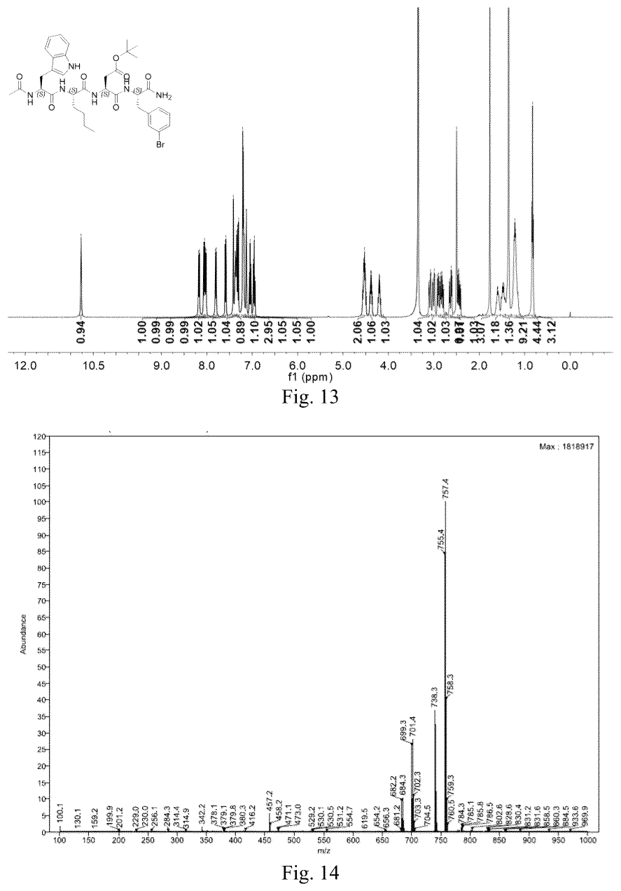

Add compound 09A (1.45 kg, 1.0 eq) and DMF (7 v) to a 10 L reaction flask and cool it to 0° C. under ice bath. Add N-methylmorpholine (1.0 eq); add acetyl-L-tryptophan (10) (1.1 eq) and stir at 0° C. for 0.5 hours; then add HOBt (1.5 eq) and EDCI (1.1 eq); stir at 25° C. for 2 hours until the HPLC reaction is complete; slowly pour the reaction solution into water (7 v), stir for 1 hour; filter, and add water (10 v) to the filter cake for 0.5 hours of mashing; filter again, and use methanol (3 v) to mash the filter cake for 0.5 hours; dry the solid, obtaining compound 11A, 1.739 kg; the purity is 97.8%. The ¹H NMR spectrum of compound 11A is shown in FIG. 13, the MS spectrum is shown in FIG. 14, ¹H NMR (DMSO-d⁶): 10.77 (1H, s), 8.17 (1H, d), 8.06 (1H, d), 8.02 (1H, d), 7.80 (1H, d), 7.58 (1H, d), 7.41 (1H, s), 7.37 (1H, m), 7.34 (1H, s), 7.31 (1H, d), 7.20 (2H, d), 7.18 (1H, s), 7.12 (1H, d), 7.04 (1H, td), 6.95 (1H, td), 4.53 (2H, m), 4.38 (1H, m), 4.19 (1H, m), 3.08 (1H, dd), 3.00 (1H, dd), 2.88 (1H, dd), 2.81 (1H, dd), 2.63 (1H, dd), 2.44 (1H, dd), 1.76 (3H, s), 1.59 (1H, m), 1.48 (1H, m), 1.35 (9H, s), 1.20 (4H, dd), 0.83 (3H, t); ESI-MS: [M+H]⁺: 755.4, 757.4.

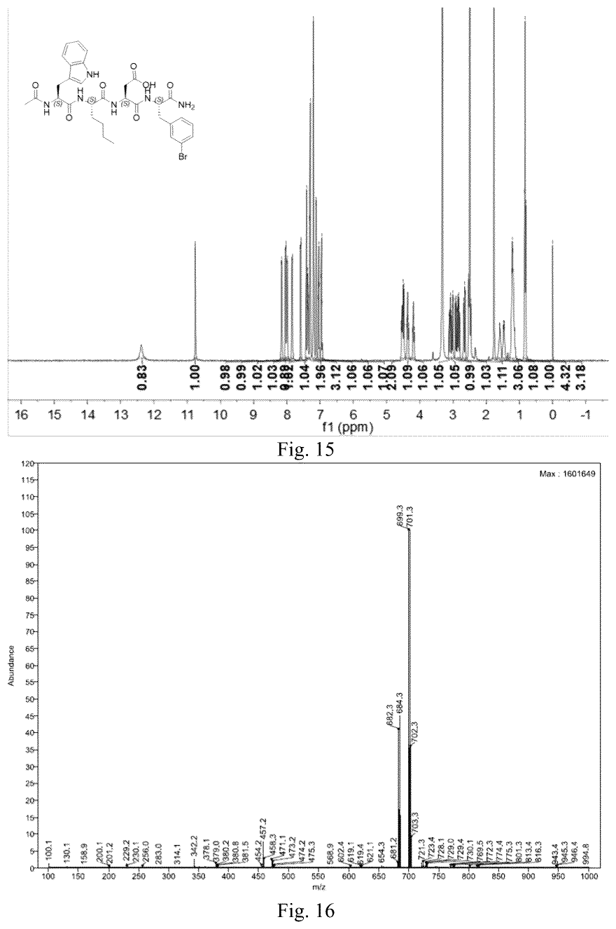

Add compound 11A (1.4 kg, 1 eq) and dichloromethane (15 V) to a 20 L jacketed flask, then cool it to 0° C. In 0° C., dissolve trimethyl iodosilane (8.0 eq) in dichloromethane (5 V), and slowly add it to the reaction mixture at a controlled temperature of 0° C. over 3 hours until complete. Maintain the reaction at 0° C. for 16 hours and monitor the reaction using HPLC. Then, add the aqueous solution of Na₂S₂O₃ (1.4 kg). After the addition is complete, continue stirring at 0° C. for 20 minutes. Add the dichloromethane solution of N-methylmorpholine (1.122 kg, 4 L). Stir for another 30 minutes. The system forms a white turbid solution. Filter, wash the filter cake twice with dichloromethane, dry, and then pulp with methanol and dichloromethane, followed by filtration, wash with dichloromethane and dry, obtaining compound 12, 1.254 g; purity 99.1%. The ¹H NMR spectrum of compound 12 is shown in FIG. 15, the MS spectrum is shown in FIG. 16, ¹H NMR (DMSO-d⁶): 12.38 (1H, s), 10.75 (1H, s), 8.17 (1H, d), 8.04 (1H, d), 8.00 (1H, d), 7.84 (1H, d), 7.59 (1H, d), 7.41 (1H, s), 7.37 (1H, m), 7.34 (1H, s), 7.31 (2H, d), 7.20 (3H, d), 7.12 (1H, s), 7.04 (1H, td), 6.95 (1H, td), 4.52 (2H, m), 4.36 (1H, m), 4.19 (1H, m), 3.09 (1H, dd), 3.02 (1H, dd), 2.90 (1H, dd), 2.82 (1H, dd), 2.66 (1H, dd), 2.50 (1H, dd), 1.77 (3H, s), 1.60 (1H, m), 1.47 (1H, m), 1.20 (4H, dd), 0.83 (3H, t); ESI-MS: [M+H]⁺: 699.3, 701.3.

### Example 2: Preparation of Compound 09 with Synthesis Route B

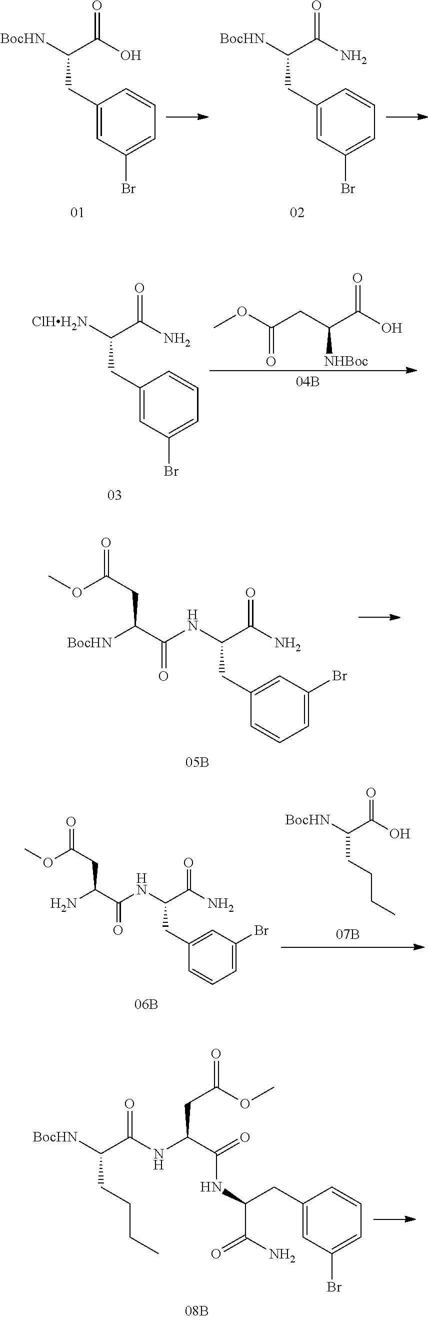

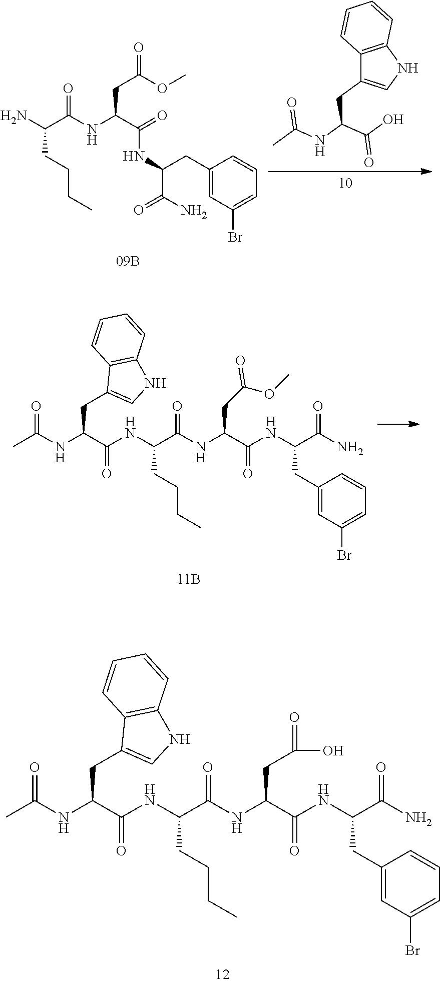

The synthesis of compounds 01, 02, 03, 05B, 08B and 11B Example 2 was carried out in accordance with the method described in Example 1.

The synthesis of compounds 06B and 09B in Example 2 was carried out with Deprotection Process I.

The reaction for preparing compound 12 from compound 11B in Example 2 was conducted under the following conditions:

| | | Solvents (volume-mass | Temperature | Reaction |
|---|---|---|---|---|
| No. | Reagents (eq.) | ratio, mL/g) | (° C.) | time (h) |
| 1 | LiOH•H₂O (2.0) | THF (14), H₂O (6) | 0-20 | 3 |
| 2 | K₂CO₃ (2.0) | THF (14), H₂O (6) | 0-20 | 3 |
| 3 | NaHCO₃ (2.0) | THF (14), H₂O (6) | 0-20 | 3 |
| 4 | Na₂CO₃ (2.0) | THF (14), H₂O (6) | 0-20 | 3 |
| 5 | Na₂HPO₄ (2.0) | THF (14), H₂O (6) | 0-20 | 3 |
| 6 | LiOH•H₂O (2.0), H₂O₂ (2.0) | THF (14), H₂O (6) | 0-20 | 16 |
| 7 | NaOH (2.0) | Toluene (10), H₂O (5) | 0-20 | 16 |

### Example 3: Preparation of Compound 09 with Synthesis Route C

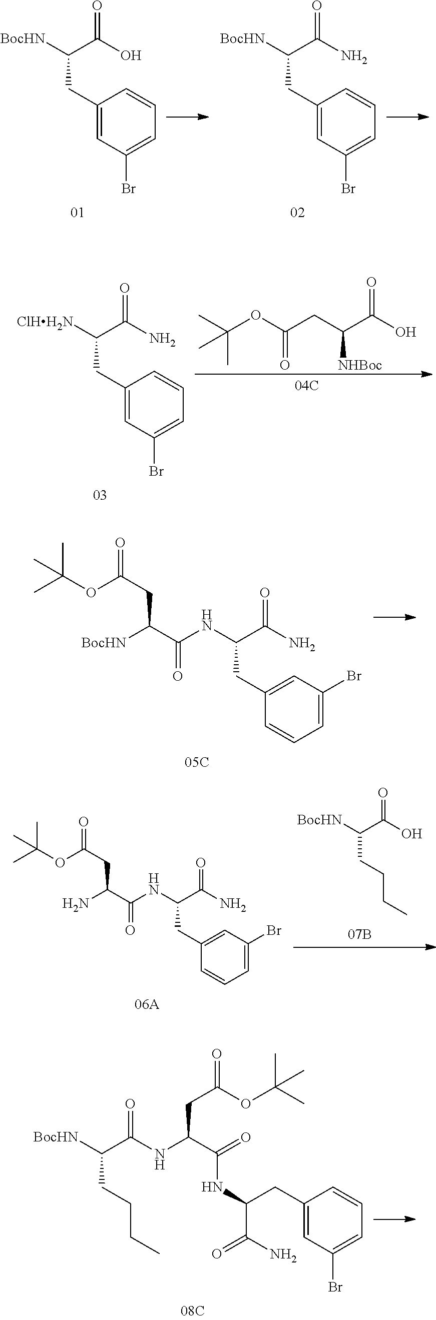

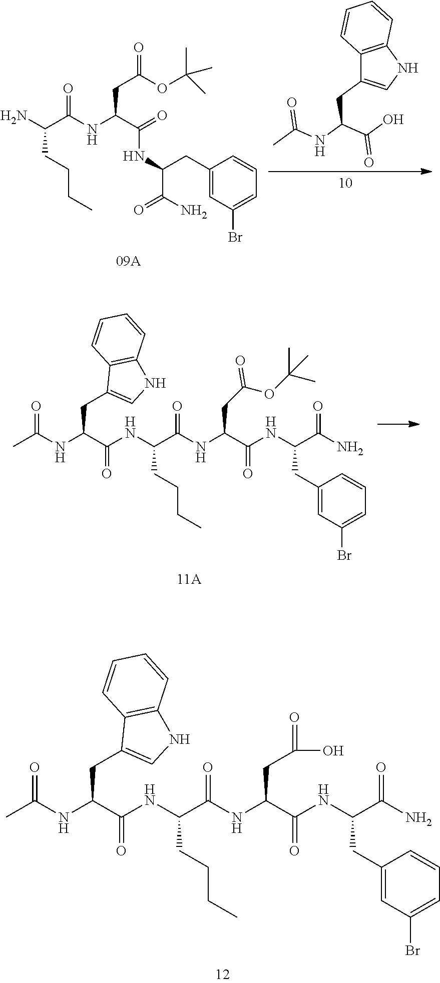

The synthesis of compounds 01, 02, 03, 05C, 08C and 11A in Example 3 was carried out in accordance with the method described in Example 1.

The synthesis of compounds compound 06A and 09A in Example 3 was carried out with Deprotection Process II.

The reaction for preparing compound 12 from compound 11A in Example 3 was conducted under the following conditions:

| | | Solvents | | | |
|---|---|---|---|---|---|
| | | (volume- | | | |
| | Reagents | mass ratio, | Temperature | Reaction | |
| No. | (eq.) | mL/g) | (° C.) | time (h) | Yields |
| 1 | HBr (3) | DMF (3.0) | 70 | 16 | 10.4% |
| 2 | TFA (3) | DMF (3.0) | 70 | 16 | 25.7% |
| 3 | HAc (3) | DMF (3.0) | 70 | 16 | 33.6% |

### Example 4: Preparation of Compound 09 with Synthesis Route D

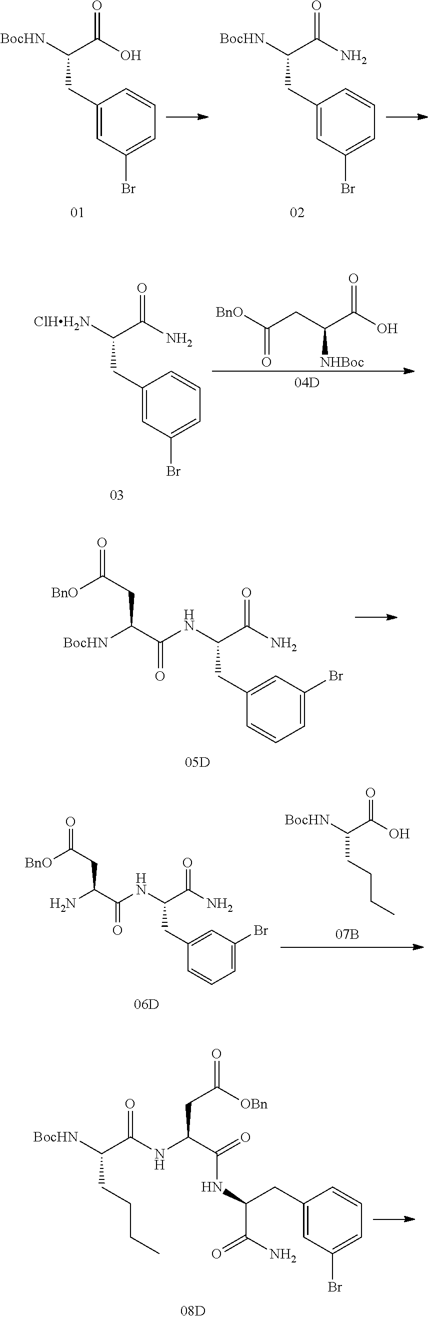

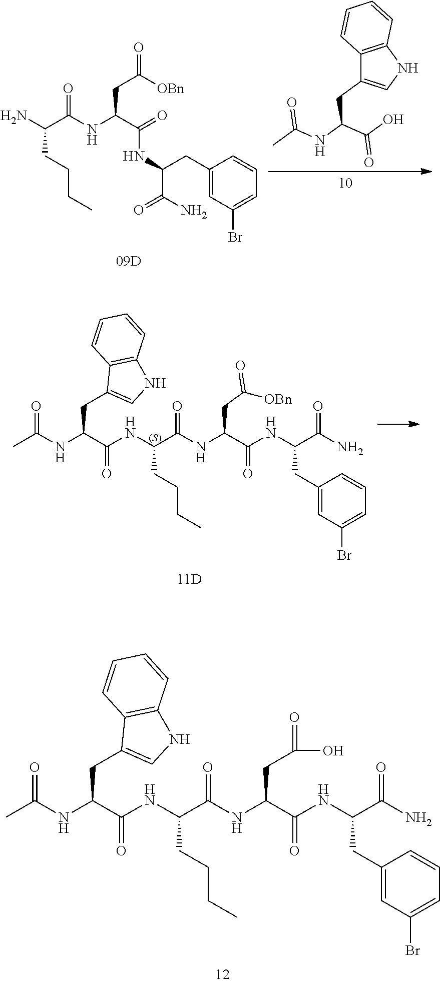

The synthesis of compounds 01, 02, 03, 05D, 08D and 11D in Example 4 was carried out in accordance with the method described in Example 1.

The synthesis of compounds compound 06D and 09D in Example 4 was carried out with Deprotection Process I.

The reaction for preparing compound 12 from compound 11D in Example 4 was conducted under the following conditions:

| | | Solvents | | | |
|---|---|---|---|---|---|
| | Reagents | (volume- | Temper- | | |
| | (11D/catalyst, | mass ratio, | ature | Reaction | |
| No. | m/m) | mL/g) | (° C.) | time (h) | Yields |
| 1 | 5% Pd/C (1:0.1) | MeOH (6.0) | 25 | 16 | 78.3% |
| 2 | 10% Pd/C (1:0.1) | EtOH (6.0) | 25 | 12 | 82.9% |
| 3 | Raney Ni (1:0.1) | MeOH (6.0) | 25 | 10 | 84.6% |

The above shows and describes the basic principle, main features and advantages of this application. Technical personnel in the industry should understand that this application is not limited by the above-mentioned embodiments. The above-mentioned embodiments and the descriptions in the specification merely illustrate the principle of this application. Without departing from the spirit and scope of this application, this application will have various changes and improvements, and all such changes and improvements fall within the protection scope of the claimed application. The protection scope of this application is defined by the appended claims and their equivalents.

What is claimed is:

1. A method for preparing a peptide compound 12 or its salt with cholecystokinin (CCK) receptor agonistic and/or antagonistic activity, comprising:
(I) condensing compound 03 or its salt with compound 04 to form compound 05;
(II) deprotecting compound 05 to yield compound 06 or its salt;
(III) condensing compound 06 or its salt with compound 07 to form compound 08;
(IV) deprotecting compound 08 to yield compound 09 or its salt;
(V) condensing compound 09 with compound 10 to form compound 11;
(VI) removing the protecting group of compound 11 to obtain compound 12 or its salt

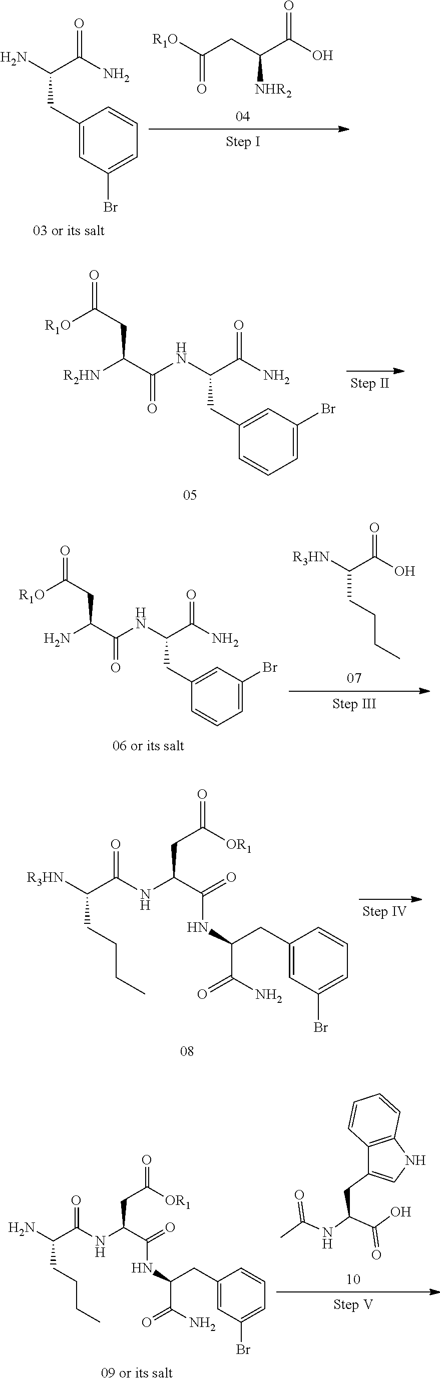

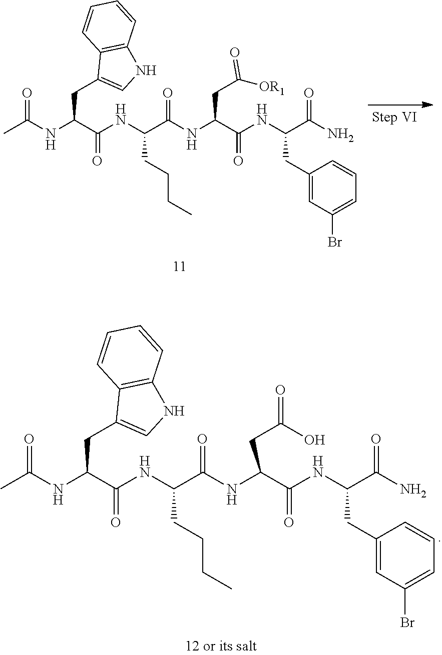

2. A pharmaceutical composition comprising the peptide compound of Formula 12 prepared according to the method of claim 1, and a pharmaceutically acceptable carrier or excipient, wherein said method comprises the steps described therein.

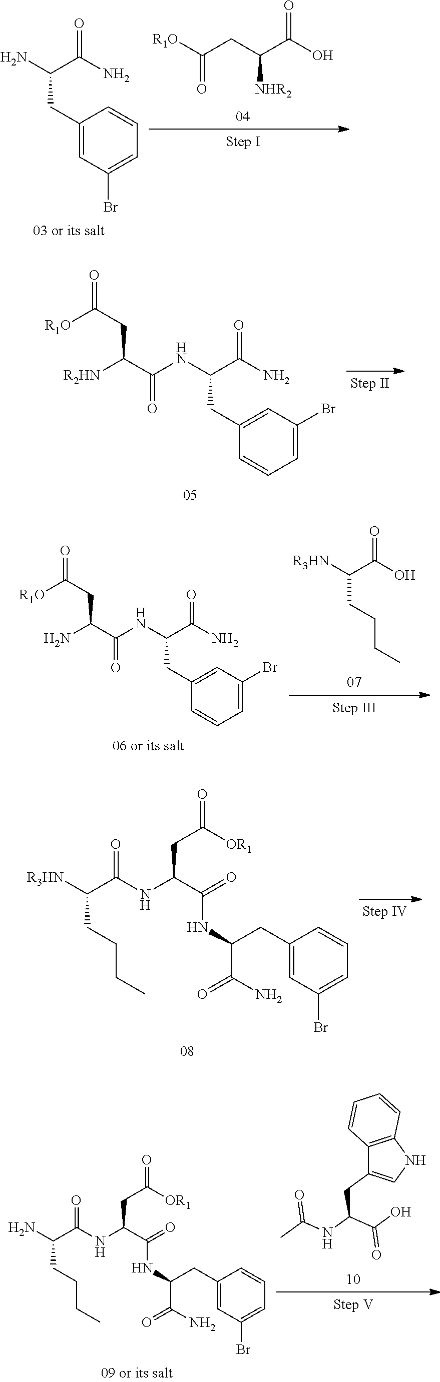

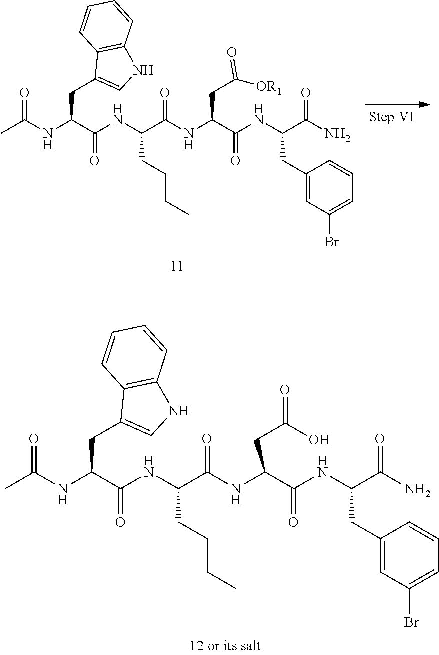

3. A pharmaceutical composition comprising the peptide compound of Formula 12 prepared according to the method of claim 1, wherein R₁ is a carboxyl protecting group, R₂ is an amino protecting group, and R₃ is an amino protecting group.

4. The method of claim 1, wherein R¹ is selected from C1-C8 alkyl or aromatic group.

5. The method of claim 4, wherein the C1-C8 alkyl is optionally substituted with one or more halogen atoms selected from fluorine, chlorine, bromine, or iodine.

6. The method of claim 4, wherein the C1-C8 alkyl is selected from methyl, ethyl, propyl, isopropyl, butyl, or tert-butyl.

7. The method of claim 1, wherein steps I, Ill and V are conducted in the presence of a coupling reagent or carboxylic acid activating agent selected from carbodiimide-based coupling agents, uronium salts, organophosphorus reagents, thionyl chloride, oxalyl chloride, phosgene, triphosgene, or cyanuric chloride; wherein the carbodiimide-based coupling agents are selected from CDI, DCC, DIC, or EDCI; wherein the uronium salts are selected from HATU, HBTU, HCTU, HAPyTU, TBTU, TSTU, TMTU, HOP, PyHOP, or PyAOP; and wherein the organophosphorus reagents are selected from DPPCI, DECP, DPPA, MPTA, or BOP-CI.

8. The method of claim 7, wherein steps I, III and V are conducted in the presence of a coupling reagent and a catalyst selected from DMAP, 4-PPY, HOAt, HOBt, HOCT, HOOBt, HOPO, NOP, HOSu, p-nitrophenol, triethylamine, diisopropylethylamine (DIEA), DBU, or pyridine.

9. The method of claim 1, wherein steps I, III and V are conducted in the presence of an inert aprotic organic solvent selected from dichloromethane (DCM), chloroform, carbon tetrachloride, 1,2-dichloroethane, DMF, DMA, DMSO, THF, benzene, toluene, acetone, or mesitylene.

10. The method of claim 1, wherein steps I, III and V are independently conducted at a temperature in the range of −30° C. to 40° C.

11. The method of claim 10, wherein steps I, III and V are independently conducted at a temperature in the range of −10° C. to 10° C. or 10° C. to 25° C.

12. The method of claim 4, wherein R¹ is selected from tert-butyl, methyl, trifluoromethyl, trichloromethyl, or benzyl, and R₂ and R₃ are independently selected from Boc, Fmoc, Teoc, Alloc, or ROCO-, wherein R in ROCO— is a C1-C8 alkyl group selected from methyl, ethyl, propyl, isopropyl, butyl, or tert-butyl.

13. The method of claim 1, wherein steps II and IV involve deprotection of amino protecting groups using a reagent selected independently from HCl, trifluoroacetic acid (TFA), methanesulfonic acid, trifluoromethanesulfonic acid, sulfuric acid, bisulfates, phosphoric acid, dihydrogen phosphates, or piperidine.

14. The method of claim 1, wherein steps II and IV are conducted in a solvent selected from water or one or more organic solvents, the organic solvent being selected from C1-C4 alkanols, C2-C6 ketones, C2-C6 ethers, C4-C8 esters, DCM, chloroform, carbon tetrachloride, 1,2-dichloroethane, DMF, DMAc, DMSO, benzene, toluene, or mesitylene.

15. The method of claim 1, wherein steps II and IV are independently carried out at a temperature in the range of −30° C. to 40° C.

16. The method of claim 1, wherein step VI involves removal of the carboxylic acid protecting group using one or more reagents selected from organic bases, inorganic bases, or trimethylsilyl iodide; wherein the organic base is selected from triethylamine, trimethylamine, diisopropylethylamine (DIEA), or pyridine; and wherein the inorganic base is selected from alkali or alkaline earth metal hydroxides, carbonates, or bicarbonates, including lithium hydroxide, sodium hydroxide, potassium hydroxide, cesium hydroxide, rubidium hydroxide, calcium hydroxide, lithium carbonate, sodium carbonate, potassium carbonate, lithium bicarbonate, sodium bicarbonate, potassium bicarbonate, sodium phosphate, potassium phosphate, or lithium phosphate.

17. The method of claim 1, wherein step VI is carried out at a temperature in the range of −30° C. to 40° C.

18. The method of claim 17, wherein step VI is conducted in water, an organic solvent, or a mixture of water and a water-miscible organic solvent; wherein the organic solvent is selected from dichloromethane, chloroform, carbon tetrachloride, or 1,2-dichloroethane.

19. The method of claim 1, further comprising the following steps.

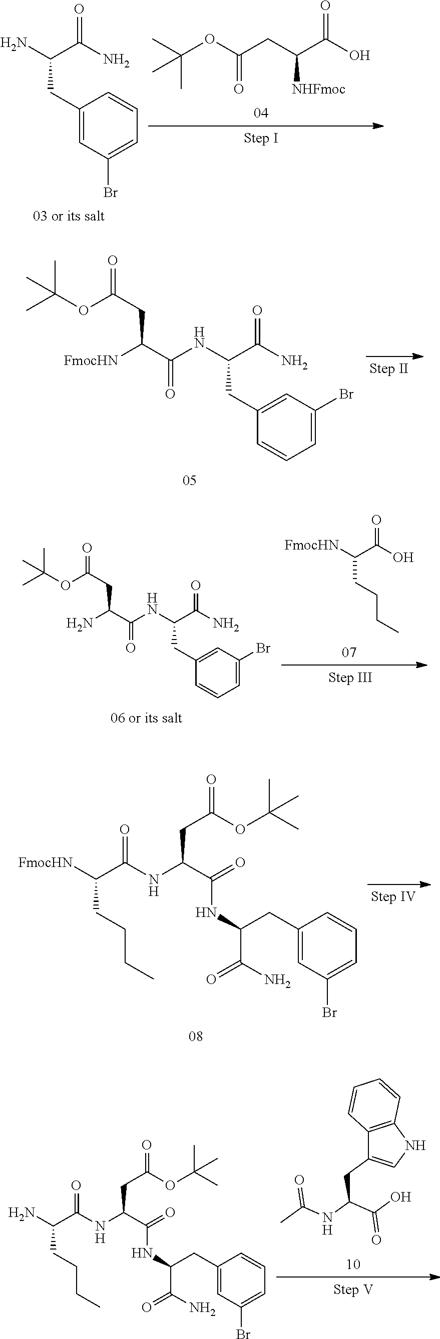

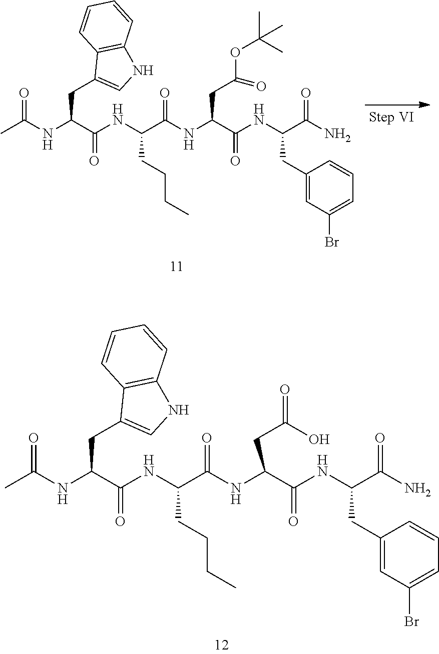 [SMILES: CCCC[C@H](NC(=O)[C@H](Cc1c[nH]c2ccccc12)NC(C)=O)C(=O)N[C@@H](CC(=O)OC(C)(C)C)C(=O)N[C@@H](Cc1cccc(Br)c1)C(N)=O ; CCCC[C@H](NC(=O)[C@H](Cc1c[nH]c2ccccc12)NC(C)=O)C(=O)N[C@@H](CC(=O)O)C(=O)N[C@@H](Cc1cccc(Br)c1)C(N)=O]

20. The method of claim 1, wherein the peptide compound of Formula 12 is obtained in a purity suitable for pharmaceutical use without the need for chromatographic purification.
